(19) United States
(12) Patent Application Publication
(10) Pub. No.: US 2014/0187634 A1
(43) Pub. Date: Jul. 3, 2014

(54) CURE FOR EDEMA OF THE FOOT AND ANKLE

(71) Applicant: AMY M. ELLIOTT, Brooklyn, NY (US)

(72) Inventor: AMY M. ELLIOTT, Brooklyn, NY (US)

(21) Appl. No.: 13/902,433

(22) Filed: May 24, 2013

Related U.S. Application Data
(60) Provisional application No. 61/848,268, filed on Dec. 31, 2012.

(51) Int. Cl. A61K 31/201; A61K 31/05

(52) U.S. Cl. CPC A61K 31/201; A61K 31/05
USPC 514/560

(57) ABSTRACT

The inventive subject matter relates to a method for treating Edema of the Foot and Ankle Edema is a medical condition characterized by swelling and discoloration caused by excess fluid trapped in the body tissues. Administration of Extra Virgin Olive Oil composition provides therapeutic results when applied to the affected area.

This application claims the benefit of U.S. Provisional Application No. 61/848,268 filed on Dec. 31, 2012, the entire contents of which are hereby incorporated.

### BACKGROUND OF THE INVENTIVE SUBJECT MATTER

1. Field of the Invention

Edema is a medical condition characterized by swelling caused by excess fluid trapped in the body tissues. Edema is the result of an imbalance in the filtration system between the capillary and interstitial spaces. Major causes of edema of the foot and ankle include venous obstruction, increased capillary permeability, and increased plasma volume secondary to sodium and water retention. Edema can affect any part of the body, but is most commonly found in the hands, arms, feet, ankles and legs.

Edema usually results from an imbalance of forces controlling fluid exchange, including an alteration in capillary hemodynamics favoring the retention of sodium and water by the kidneys and the movement of fluid from the vascular space into the interstitium. Edema occurs when forces such as an elevation in capillary hydraulic pressure, an increase in capillary permeability or interstitial oncotic pressure, or a reduction in plasma oncotic pressure increase net filtration. The kidneys control body fluid homeostasis, and body water is primarily regulated by antidiuretic hormone which is secreted in response to changes in blood volume, tonicity, and blood pressure. Sodium and other ion concentrations in extracellular fluid are maintained within a narrow range, so extracellular fluid volume can be maintained at a constant level despite wide fluctuations in intake. Whatever the cause, edema persists because of compensatory mechanisms geared toward maintaining plasma volume.

2. Description of Related Art

Treatment of Edema known in the art includes reversing the underlying disorder, treatment with diuretics, and restricting dietary sodium to minimize fluid retention. Drug treatment is not required to treat edema in all patients, and in some patients, sufficient sodium restriction and elevation of the lower extremities above the level of the left atrium are effective. However, drug treatment is required for most patients in addition to nonpharmacological treatments. Diuretics exert their effects at different sites in the kidneys. Different diuretics exhibit different absorption rates and half-lives which may result in treatment failure.

Diuretic resistance is a failure to create negative sodium balance despite the use of high dosages of diuretics. Resistance should be suspected in patients with persistent edema who are receiving appropriate diuretic treatment and restricting physical activity and sodium intake. The problem of diuretic resistance generally will not be solved without an understanding of the pharmacokinetics of each diuretic agent used. Additionally, treatment failure may result due to nonadherence to therapy. This is a concern with older adults who are taking multiple medications and are at risk for urinary incontinence, and therefore limit use of diuretics to maintain continence. Diuretic resistance can be aggravated by the concamitant use of nonsteroidal anti-inflammatory drugs (NSAIDs), which may negate the diuretic effect. Tolerance to diuretics may also cause treatment failure.

### SUMMARY OF THE INVENTIVE SUBJECT MATTER

The present inventive subject matter relates to a method for treating Edema in a patient, comprising the step of administering a therapeutically effective amount of Extra Virgin Olive Oil.

### DETAILED DESCRIPTION OF THE INVENTION

The term “therapeutically effective amount” as used herein refers to that amount of extra virgin olive oil which will reduce Edema. The term “Edema” as used herein refers broadly to a medical condition characterized by swelling caused by excess fluid trapped in the body's tissues.

The olive tree, Olea europaea, produces the olive fruit and is native to the Mediterranean basin and parts of Asia Minor. Historically, the products of the olive tree have been used as aphrodisiacs, emollients, laxatives, nutritives, sedatives, and tonics. Specific conditions traditionally treated include colic, alopecia, paralysis, rheumatic pain, sciatica, and hypertension. To manufacture olive oil, olives are crushed to create pomace, which is then homogenized before being pressed to produce oil. The first oil extracted is the high quality extra virgin olive oil. The pomace can then be processed again to yield the lower quality refined virgin olive oil. Olive oil is approximately 72-percent oleic acid, which has been reported to play a role in cancer prevention. A range phenols in olive oil provides additional health benefits. Some of the main phenols in olive oil are catechols, and it has been known for many years that compounds with a catechol group exhibit antioxidant activity. Epidemiological studies have shown that dietary olive oil reduce the incidence of coronary heart disease, hypertension, and cancer.

The inventive subject matter introduces an additional health benefit of extra virgin olive oil. This additional health benefit is the treatment of edema. Edema is treated by the topical application of extra virgin olive oil on the skin of the ankle and the foot. Topical application of 5-15 milliliters of extra virgin olive oil produces the best results.

1. A method for treating edema of the foot and ankle comprising topical administration of a therapeutically effective amount of extra virgin olive oil on the foot and ankle.

2. The method of claim 1, wherein 5-15 milliliters of extra virgin olive oil is topically applied.

3. The method of claim 1, wherein the extra virgin olive oil is manufactured in Greece.
